(19) United States
(12) Patent Application Publication
(10) Pub. No.: US 2022/0096369 A1
(43) Pub. Date: Mar. 31, 2022

(54) COMPOSITIONS COMPRISING CANNABINOIDS AND METHODS OF USE THEREOF

(71) Applicant: Irwin C. Jacobs, Defiance, MO (US)

(72) Inventor: Irwin C. Jacobs, Defiance, MO (US)

(21) Appl. No.: 17/486,123

(22) Filed: Sep. 27, 2021

Related U.S. Application Data
(60) Provisional application No. 63/083,538, filed on Sep. 25, 2020.

(51) Int. Cl. A61K 9/00; A61K 47/46; A61K 47/10; A61K 47/14; A61K 47/24; A61K 31/05

(52) U.S. Cl. CPC A61K 9/0014; A61K 47/46; A61K 31/05; A61K 47/14; A61K 47/24; A61K 47/10

(57) ABSTRACT

The present disclosure is related to transdermal cannabinoid compositions and methods of use thereof.

### CROSS-REFERENCE TO RELATED APPLICATIONS

This application claims priority from Provisional Application No. 63/083,538, filed Sep. 25, 2020, the entire contents of which are hereby incorporated by reference.

### FIELD OF THE DISCLOSURE

The present disclosure is related to transdermal cannabinoid compositions and methods of use thereof.

### BACKGROUND OF THE DISCLOSURE

Cannabidiol (CBD) is a phytocannabinoid from the Cannabis hemp plant, Cannabis sativa. Cannabidiol is not psychoactive; that is, it will not produce a “high”, but is believed to have beneficial effects including as an anti-anxiety agent, a sleep aid, and as a pain reliever (Velasquez-Manoff, 2019). CBD has been proven, through clinical trials, to have a positive effect on preventing childhood seizures from two specific epileptic syndromes; Dravet syndrome and Lennox-Gastaut syndrome (Grinspoon, 2018).

Products currently available are often prepared using the oil derived from the hemp plant and contain non-standardized low levels of CBD often in the 2 to 4% range or higher purity CBD extracts diluted with varying plant oils or medium chain triglycerides. The oils do not lend themselves to reproducible application on the skin because of its low viscosity and unreliable content, or the consumer is directed to ingest the oils. Oral CBD has long been known to have very low oral bioavailability and is generally metabolized before reaching the blood stream. Hence, there is a need in the art for a consumer friendly reproducibly bioavailable form of a cannabinoid.

### SUMMARY OF THE DISCLOSURE

One aspect of the present disclosure encompasses a transdermal cannabinoid composition, the composition comprising up to about 20% of one or more cannabinoid, from about 4 to about 15% phosphatidyl choline, and no fatty acids.

In some aspects of the present disclosure the transdermal composition may further comprise glycerides.

Some aspects of the present disclosure encompasses a transdermal cannabinoid composition, wherein the composition comprises up to about 20% oleyl alcohol, up to about 30% glycerides, up to about 15% propylene glycol monocaprylate, up to about 5% chamomile, and up to about 30% PEG or PEG/PPG/PEG block copolymers (polyethylene oxide/polypropylene oxide/polyethylene oxide block co polymer).

In some aspects the present disclosure also encompasses methods of transdermally delivering a cannabinoid. Other aspects and iterations are disclosed herein.

### DESCRIPTION OF THE FIGURES

FIG. 1 depicts a graph showing the flux defined as the mass of CBD moving through the surface area of a model membrane over time for formulations B, D, E and F.

FIG. 2 depicts a graph showing the cumulative amount of CBD that permeated the membrane over time for formulations B, D, E and F. One can note the significantly higher absorption in formulations B and D as compared to the system E without the phosphatidyl choline and system F representative of currently available technology.

### DETAILED DESCRIPTION

The present disclosure is directed to transdermal cannabinoid compositions and methods of use thereof. Each are described in more detail below.

### I. Compositions

One aspect of the present disclosure encompasses a transdermal cannabinoid composition. Generally speaking, the composition comprises up to about 20% of one or more cannabinoids, from about 4 to about 15% phosphatidyl choline, and no fatty acids. As used herein, a “fatty acid” is defined as a carboxylic acid consisting of a hydrocarbon chain and a terminal carboxyl group.

### (a) One or More Cannabinoid

A transdermal cannabinoid composition of the present disclosure comprises one or more cannabinoids. As used herein, “cannabinoid” includes any compound that interacts with a cannabinoid receptor and various cannabinoid mimetics. Suitable cannabinoids may include major cannabinoids or minor cannabinoids. In all instances, cannabinoids used in the present disclosure may be isolated from natural sources or synthetically manufactured. Non-limiting examples of major cannabinoids include D-9THC (THC) and cannabidiol (CBD). Non-limiting sources of minor cannabinoids may include CBGV (cannabigerovarin), CBGVA (cannabigerovarinic acid), CBG (cannabigerol), CBGA (cannbigerolic acid), Cannabinerolic acid, Cannabinerol, CBN (cannabinol), CBNA (cannabinolic acid), THCV (tetrahydrocannabivarin), THCA (tetrahydrocannabinolic acid), D8-THC (delta-8-tetrahydrocannabinol), CBDA (cannabidiolic acid), CBC (cannabichromene), CBCA, (cannabichromenic acid), CBDV (cannabivarin), CBL (Cannabicyclol), CBLA (Cannabicyclolic acid), Cannabicyclovarin, CBE (cannabielsoin), CBF (cannabifuran), Cannabicitran, Cannabitriol, and Cannabiorcol. In exemplary embodiments, a transdermal cannabinoid composition of the present disclosure comprises CBD.

In some embodiments, a transdermal cannabinoid composition of the present disclosure may comprise one or more of the group of minor cannabinoids consisting of CBGV (cannabigerovarin), CBGVA (cannabigerovarinic acid), CBG (cannabigerol), CBGA (cannbigerolic acid), Cannabinerolic acid, Cannabinerol, CBN (cannabinol), CBNA (cannabinolic acid), THCV (tetrahydrocannabivarin), THCA (tetrahydrocannabinolic acid), D8-THC (delta-8-tetrahydrocannabinol), CBDA (cannabidiolic acid), CBC (cannabichromene), CBCA, (cannabichromenic acid), CBDV (cannabivarin), CBL (Cannabicyclol), CBLA (Cannabicyclolic acid), Cannabicyclovarin, CBE (cannabielsoin), CBF (cannabifuran), Cannabicitran, Cannabitriol, and Cannabiorcol. In another embodiment, a transdermal cannabinoid composition of the present disclosure may comprise two or more, three or more, four or more, five or more, six or more, seven or more, eight or more, nine or more, or ten or more of the group of minor cannabinoids consisting of CBGV (cannabigerovarin), CBGVA (cannabigerovarinic acid), CBG (cannabigerol), CBGA (cannbigerolic acid), Cannabinerolic acid, Cannabinerol, CBN (cannabinol), CBNA (cannabinolic acid), THCV (tetrahydrocannabivarin), THCA (tetrahydrocannabinolic acid), D8-THC (delta-8-tetrahydrocannabinol), CBDA (cannabidiolic acid), CBC (cannabichromene), CBCA, (cannabichromenic acid), CBDV (cannabivarin), CBL (Cannabicyclol), CBLA (Cannabicyclolic acid), Cannabicyclovarin, CBE (cannabielsoin), CBF (cannabifuran), Cannabicitran, Cannabitriol, and Cannabiorcol. In yet another embodiment, a transdermal cannabinoid composition of the present disclosure may comprise at least 11, at least 12, at least 13, at least 14, at least 15, at least 16, at least 17, at least 18, at least 19, at least 20, at least 21 or at least 22 or more of the group of minor cannabinoids consisting of CBGV (cannabigerovarin), CBGVA (cannabigerovarinic acid), CBG (cannabigerol), CBGA (cannbigerolic acid), Cannabinerolic acid, Cannabinerol, CBN (cannabinol), CBNA (cannabinolic acid), THCV (tetrahydrocannabivarin), THCA (tetrahydrocannabinolic acid), D8-THC (delta-8-tetrahydrocannabinol), CBDA (cannabidiolic acid), CBC (cannabichromene), CBCA, (cannabichromenic acid), CBDV (cannabivarin), CBL (Cannabicyclol), CBLA (Cannabicyclolic acid), Cannabicyclovarin, CBE (cannabielsoin), CBF (cannabifuran), Cannabicitran, Cannabitriol, and Cannabiorcol. In each of the above embodiments, a transdermal cannabinoid composition of the present disclosure may also comprise CBD.

A transdermal cannabinoid composition of the disclosure may comprise up to about 20% of one or more cannabinoids. For instance, a transdermal cannabinoid composition may comprise about 1, 2, 3, 4, 5, 6, 7, 8, 9, 10, 11, 12, 13, 14, 15, 16, 17, 18, 19, or 20% of one or more cannabinoids. In some embodiments, a transdermal cannabinoid composition may comprise about 1 to about 20, about 5 to about 15, about 5 to about 10, about 10 to about 15, or about 10 to about 20% of one or more cannabinoids.

In particular embodiments, a transdermal cannabinoid composition of the disclosure may comprise up to about 20% CBD, and one or more additional cannabinoids. For instance, a transdermal cannabinoid composition may comprise about 1, 2, 3, 4, 5, 6, 7, 8, 9, 10, 11, 12, 13, 14, 15, 16, 17, 18, 19, or 20% of CBD and one or more additional cannabinoids. In some embodiments, a transdermal cannabinoid composition may comprise about 1 to about 20, about 5 to about 15, about 5 to about 10, about 10 to about 15, or about 10 to about 20% of CBD and one or more additional cannabinoids.

In other embodiments, a transdermal cannabinoid composition of the disclosure may comprise up to about 20% of CBD, and no additional cannabinoids. For instance, a transdermal cannabinoid composition may comprise about 1, 2, 3, 4, 5, 6, 7, 8, 9, 10, 11, 12, 13, 14, 15, 16, 17, 18, 19, or 20% of CBD and no additional cannabinoids. In some embodiments, a transdermal cannabinoid composition may comprise about 1 to about 20, about 5 to about 15, about 5 to about 10, about 10 to about 15, or about 10 to about 20% of CBD and no additional cannabinoids.

Generally speaking, a transdermal cannabinoid composition of the present disclosure may be used to deliver about 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, 2000, 2100, 2200, 2300, or 2400 mg/day of a cannabinoid; in exemplary embodiments, of CBD. These amounts may be comprised in a single dose, or more than one dose (e.g. two, three, or more than three doses).

### (b) Phosphatidyl Choline

A transdermal cannabinoid composition of the present disclosure also encompasses phosphatidyl choline. As used herein, “phosphatidyl choline” refers to a phospholipid composed of a choline head group and glycerophosphoric acid. Phosphatidyl choline may be naturally sourced or synthetically produced. In some embodiments, a transdermal cannabinoid composition described herein may encompass phosphatidyl choline enriched lecithin. A composition of the present disclosure may comprise up to about 15% phosphatidyl choline. For instance, a composition may comprise about 1, 2, 3, 4, 5, 6, 7, 8, 9, 10, 11, 12, 13, 14, or 15% phosphatidyl choline. In some embodiments, a composition may comprise about 4 to about 15% phosphatidyl choline. In certain embodiments, a composition may comprise about 4 to about 10% phosphatidyl choline. In still other embodiments, a composition may comprise about 5 to about 8% phosphatidyl choline.

### (c) Glycerides

A transdermal cannabinoid composition of the present disclosure may also encompass one or more glycerides. In some embodiments, the glycerides are monoglycerides. In other embodiments, the glycerides are diglycerides. In certain embodiments, the glycerides are a combination of diglycerides and monoglycerides. In particular embodiments, the glycerides may have medium or long alkyl chains. In some particular embodiments, the glycerides may be polyoxyl derivatives of medium or long chain alkyl glycerides.

A composition of the present disclosure may comprise up to about 30% of one or more glycerides. For instance, a composition may comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30% of one or more glycerides. In some embodiments, a composition may comprise about 5 to about 30% of one or more glycerides. In certain embodiments, a composition may comprise about 10 to about 25% of one or more glycerides. In still other embodiments, a composition may comprise about 12 to about 22% of one or more glycerides.

In particular embodiments, a cannabinoid composition of the present disclosure may comprise up to about 30% of caprylocaproyl polyoxyl 8 glycerides. For instance, a cannabinoid composition of the present disclosure may comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30% of caprylocaproyl polyoxyl 8 glycerides. Non-limiting examples of a commercial product comprising caprylocaproyl polyoxyl 8 glycerides includes Labrasol ALF.

In some embodiments, a cannabinoid composition of the present disclosure may comprise up to about 30% of oleyl polyoxyl 6 glycerides. For instance, a cannabinoid composition of the present disclosure may comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30% of oleyl polyoxyl 6 glycerides. Non-limiting examples of a commercial product comprising oleyl polyoxyl 6 glycerides includes Labrafil M1944CS.

In preferred embodiments, a cannabinoid composition of the present disclosure may comprise up to about 30% of caprylocaproyl polyoxyl 8 glycerides and oleyl polyoxyl 6 glycerides.

### (d) Alcohols

A transdermal cannabinoid composition of the present disclosure may also encompass one or more alcohols. As used herein, “alcohol” refers to a molecule with at least one terminal OH group.

A composition of the present disclosure may comprise up to about 20% of one or more alcohols. For instance, a composition may comprise about 0, 1, 2, 3, 4, 5, 6, 7, 8, 9, 10, 11, 12, 13, 14, 15, 16, 17, 18, 19, or 20% of one or more alcohols. In some embodiments, a composition may comprise about 2 to about 15% of one or more alcohols. In certain embodiments, a composition may comprise about 10 to about 20% of one or more alcohols. In still other embodiments, a composition may comprise about 5 to about 18% of one or more alcohols.

In some embodiments, a transdermal cannabinoid composition of the present disclosure may comprise propylene glycol monocaprylate. For instance, a composition may comprise about 1, 2, 3, 4, 5, 6, 7, 8, 9, 10, 11, 12, 13, 14, 15, 16, 17, 18, 19, or 20% of propylene glycol monocaprylate. Non-limiting examples of commercial sources of propylene glycol monocaprylate include Capryol 90.

In certain embodiments, a transdermal cannabinoid composition of the present disclosure may comprise diethylene glycol monoethyl ether. For instance, a composition may comprise about 1, 2, 3, 4, 5, 6, 7, 8, 9, 10, 11, 12, 13, 14, 15, 16, 17, 18, 19, or 20% of diethylene glycol monoethyl ether. Non-limiting examples of commercial sources of diethylene glycol monoethyl include Transcutol.

In still other embodiments, a transdermal cannabinoid composition of the present disclosure may comprise oleyl alcohol. For instance, a composition may comprise about 0, 1, 2, 3, 4, 5, 6, 7, 8, 9, 10, 11, 12, 13, 14, 15, 16, 17, 18, 19, or 20% of oleyl alcohol.

In particular embodiments, a transdermal cannabinoid composition of the present disclosure may comprise up to about 30% of any combination of propylene glycol monocaprylate, diethylene glycol monoethyl ether, and oleyl alcohol.

### (e) Other Components

A transdermal cannabinoid composition of the present disclosure may further comprise one or more additional components. Generally speaking, the one or more additional components may comprise from 0% to about 99% of a transdermal cannabinoid composition of the present disclosure by weight. For instance, a transdermal cannabinoid composition may comprise about 5, 10, 15, 20, 25, 30, 35, 40, 45, 50, 55, 60, 65, 70, 75, 80, 85, 90, or 95% of one or more additional components. In some embodiments, a transdermal cannabinoid composition may comprise about 1 to about 25, about 15 to about 35, about 20 to about 40, about 25 to about 45, about 30 to about 50, about 35 to about 55, about 40 to about 60, about 45 to about 65, about 50 to about 70, about 55 to about 75, about 60 to about 80, about 65 to about 85, about 70 to about 90, about 75 to about 95, or about 80 to about 99% of one or more additional components.

Non-limiting examples of additional components may include PEG or PEG like polymers, chamomile, terpenes or terpenoids, flavinoids and isopropyl myristate.

i. PEG

In some embodiments, a transdermal cannabinoid composition of the present disclosure comprises PEG or a PEG like polymer, for instance, a polyethylene oxide/polypropylene oxide/polyethylene oxide glycol block copolymer. A transdermal cannabinoid composition of the present disclosure may comprise up to about 40% of such a component, for instance, about 5, 10, 15, 20, 25, 30, 35, or 40%. Non-limiting examples of commercially available polymers include Kollisolve P237.

ii. Chamomile

In some embodiments, a transdermal cannabinoid composition of the present disclosure comprises an amount of an essential oil such as chamomile. A transdermal cannabinoid composition of the present disclosure may comprise up to about 15% of such a component, for instance, about 1, 2, 3, 4, 5, 6, 7, 8, 9, 10, 11, 12, 13, 14, or 15%.

Other suitable herbs or plant extracts may also be used, in addition to or in place of chamomile oil. Non-limiting examples of such oils include cedarwood, oil lavender oil, rosemary oil, lemon oil, cypress oil, Ylang Ylang oil, tea tree oil, peppermint oil, ginger oil, geranium oil, patchouli oil, bergamot oil, and frankincense oil.

iii. Isopropyl Myristate

In some embodiments, a transdermal cannabinoid composition of the present disclosure comprises isopropyl myristate. A transdermal cannabinoid composition of the present disclosure may comprise up to about 40% of such a component, for instance, about 5, 10, 15, 20, 25, 30, 35, or 40%.

### (f) Purity

Generally speaking, a compound included in a transdermal cannabinoid composition of the disclosure, such as a minor cannabinoid or a major cannabinoid should be of high purity and consistent quality, regardless of source. For instance, a minor or major cannabinoid used in a composition of the disclosure is at least 85, 86, 87, 88, 89, 90, 91, 92, 93, 94, 95, 96, 97, 98 or 99% pure. In certain embodiments, a minor or major cannabinoid used in a composition of the disclosure is at least 95, 96, 97, 98, or 99% pure. In preferred embodiments, a minor or major cannabinoid used in a composition of the disclosure is at least 97% pure.

Manufacture of a compound for a composition of the disclosure is preferred to meet GLP requirements. In certain embodiments, manufacture of a compound for a composition of the disclosure meets GMP requirements to ensure consistent quality.

Quantitative/qualitative methods to confirm quality and purity include use of High Performance Liquid Chromatography (HPLC), Ultra Performance Liquid Chromatography (UPLC), Nuclear Magnetic Resonance (NMR) spectroscopy, Gas Chromatography (GC), Thin Layer Chromatography (TLC), and standard methods for testing for contamination of microbiological, heavy metal, pesticide, or other contaminants.

### (g) Enumerated Embodiments

In one embodiment, a transdermal cannabinoid composition of the present disclosure comprises up to about 20% of one or more cannabinoid, up to about 15% phosphatidyl choline, no fatty acids, up to about 40% of one or more alcohol, up to about 30% glycerides, and up to about 30% of PEG or a PEG like polymer.

In another embodiment, a transdermal cannabinoid composition of the present disclosure comprises up to about 20% of one or more cannabinoid, up to about 15% phosphatidyl choline, no fatty acids, up to about 40% of one or more alcohol, up to about 30% glycerides, and up to about 20% chamomile.

In yet another embodiment, a transdermal cannabinoid composition of the present disclosure comprises up to about 20% of one or more cannabinoid, up to about 15% phosphatidyl choline, no fatty acids, up to about 40% of one or more alcohol, up to about 30% glycerides, and up to about 30% isopropyl myristate.

In still another embodiment, a transdermal cannabinoid composition of the present disclosure comprises up to about 20% of one or more cannabinoid, up to about 15% phosphatidyl choline, no fatty acids, up to about 40% of one or more alcohol, up to about 30% glycerides, up to about 30% of PEG or a PEG like polymer, up to about 30% isopropyl myristate and up to about 20% chamomile.

In yet still another embodiment, a transdermal cannabinoid composition of the present disclosure comprises up to about 20% of CBD, up to about 15% phosphatidyl choline, no fatty acids, up to about 40% of one or more alcohol, up to about 30% glycerides, and up to about 30% of PEG or a PEG like polymer.

In some embodiments, a transdermal cannabinoid composition of the present disclosure comprises up to about 20% of CBD, up to about 15% phosphatidyl choline, no fatty acids, up to about 40% of one or more alcohol, up to about 30% glycerides, and up to about 20% chamomile.

In other embodiments, a transdermal cannabinoid composition of the present disclosure comprises up to about 20% of CBD, up to about 15% phosphatidyl choline, no fatty acids, up to about 40% of one or more alcohol, up to about 30% glycerides, and up to about 30% isopropyl myristate.

In particular embodiments, a transdermal cannabinoid composition of the present disclosure comprises up to about 20% CBD, between about 4 to about 15% phosphatidyl choline, no fatty acids, up to about 40% of one or more alcohol, up to about 30% glycerides, up to about 30% of PEG or a PEG like polymer, up to about 30% isopropyl myristate and up to about 20% chamomile.

In additional embodiments, a transdermal cannabinoid composition of the present disclosure comprises up to about 20% of CBD, between about 4 to about 15% phosphatidyl choline, no fatty acids, up to about 40% of one or more alcohol, up to about 30% glycerides, and up to about 30% of PEG or a PEG like polymer.

In another additional embodiment, a transdermal cannabinoid composition of the present disclosure comprises up to about 20% of CBD, between about 4 to about 15% phosphatidyl choline, no fatty acids, up to about 40% of one or more alcohol, up to about 30% glycerides, and up to about 20% chamomile.

In still another additional embodiment, a transdermal cannabinoid composition of the present disclosure comprises up to about 20% of CBD, between about 4 to about 15% phosphatidyl choline, no fatty acids, up to about 40% of one or more alcohol, up to about 30% glycerides, and up to about 30% isopropyl myristate.

In yet another additional embodiment, a transdermal cannabinoid composition of the present disclosure comprises up to about 20% CBD, between about 4 to about 15% phosphatidyl choline, no fatty acids, up to about 40% of one or more alcohol, up to about 30% glycerides, up to about 30% of PEG or a PEG like polymer, up to about 30% isopropyl myristate and up to about 20% chamomile.

In still yet another additional embodiment, a transdermal cannabinoid composition of the present disclosure comprises between about 5 to about 20% CBD, between about 4 to about 15% phosphatidyl choline, no fatty acids, up to about 40% of one or more alcohol, up to about 30% glycerides, up to about 30% of PEG or a PEG like polymer, up to about 30% isopropyl myristate and up to about 20% chamomile.

In certain embodiments, a transdermal cannabinoid composition of the present disclosure comprises between about 5 to about 20% of CBD, between about 4 to about 15% phosphatidyl choline, no fatty acids, up to about 40% of one or more alcohol, up to about 30% glycerides, and up to about 30% of PEG or a PEG like polymer.

In particular embodiments, a transdermal cannabinoid composition of the present disclosure comprises between about 5 to about 20% of CBD, between about 4 to about 15% phosphatidyl choline, no fatty acids, up to about 40% of one or more alcohol, up to about 30% glycerides, and up to about 20% chamomile.

In certain particular embodiments, a transdermal cannabinoid composition of the present disclosure comprises between about 5 to about 20% of CBD, between about 4 to about 15% phosphatidyl choline, no fatty acids, up to about 40% of one or more alcohol, up to about 30% glycerides, and up to about 30% isopropyl myristate.

In other embodiments, a transdermal cannabinoid composition of the present disclosure comprises between about 5 to about 20% CBD, between about 4 to about 15% phosphatidyl choline, no fatty acids, up to about 40% of one or more alcohol selected from the group consisting of oleyl alcohol, diethylene glycol monoethyl ether, and propylene glycol monocaprylate, up to about 30% glycerides selected from the group consisting of caprylocaproyl polyoxyl 8 glycerides and oleyl polyoxyl 6 glycerides, up to about 30% of PEG or a PEG like polymer, up to about 25% isopropyl myristate and up to about 10% chamomile.

In yet other embodiments, a transdermal cannabinoid composition of the present disclosure comprises between about 5 to about 20% of CBD, between about 4 to about 15% phosphatidyl choline, no fatty acids, up to about 40% of one or more alcohol selected from the group consisting of oleyl alcohol, diethylene glycol monoethyl ether, and propylene glycol monocaprylate, up to about 30% glycerides selected from the group consisting of caprylocaproyl polyoxyl 8 glycerides and oleyl polyoxyl 6 glycerides, and up to about 30% of PEG or a PEG like polymer.

In still other embodiments, a transdermal cannabinoid composition of the present disclosure comprises between about 5 to about 20% of CBD, between about 4 to about 15% phosphatidyl choline, no fatty acids, up to about 40% of one or more alcohol selected from the group consisting of oleyl alcohol, diethylene glycol monoethyl ether, and propylene glycol monocaprylate, up to about 30% glycerides selected from the group consisting of caprylocaproyl polyoxyl 8 glycerides and oleyl polyoxyl 6 glycerides, and up to about 10% chamomile.

In yet still other embodiments, a transdermal cannabinoid composition of the present disclosure comprises between about 5 to about 20% of CBD, between about 4 to about 15% phosphatidyl choline, no fatty acids, up to about 40% of one or more alcohol selected from the group consisting of oleyl alcohol, diethylene glycol monoethyl ether, and propylene glycol monocaprylate, up to about 30% glycerides selected from the group consisting of caprylocaproyl polyoxyl 8 glycerides and oleyl polyoxyl 6 glycerides, and up to about 25% isopropyl myristate.

In certain embodiments, a transdermal cannabinoid composition of the present disclosure comprises between about 5 to about 15% CBD, between about 5 to about 10% phosphatidyl choline, no fatty acids, up to about 20% oleyl alcohol, up to about 12% diethylene glycol monoethyl ether, up to about 10% propylene glycol monocaprylate, up to about 20% caprylocaproyl polyoxyl 8 glycerides, up to about 8% oleyl polyoxyl 6 glycerides, up to about 30% of PEG or a PEG like polymer, up to about 25% isopropyl myristate and up to about 5% chamomile.

In exemplary embodiments, a transdermal cannabinoid composition of the present disclosure comprises between about 5 to about 15% CBD, between about 5 to about 10% phosphatidyl choline, no fatty acids, up to about 12% diethylene glycol monoethyl ether, up to about 10% propylene glycol monocaprylate, up to about 20% caprylocaproyl polyoxyl 8 glycerides, up to about 8% oleyl polyoxyl 6 glycerides, up to about 30% of PEG or a PEG like polymer, up to about 25% isopropyl myristate and up to about 5% chamomile.

In other exemplary embodiments, a transdermal cannabinoid composition of the present disclosure comprises between about 5 to about 15% CBD, between about 5 to about 10% phosphatidyl choline, no fatty acids, up to about 20% oleyl alcohol, up to about 10% propylene glycol monocaprylate, up to about 20% caprylocaproyl polyoxyl 8 glycerides, up to about 30% of PEG or a PEG like polymer, up to about 25% isopropyl myristate and up to about 5% chamomile.

### II. Methods

Another aspect of the present disclosure encompasses methods of using transdermal cannabinoid compositions.

A transdermal cannabinoid composition of the present disclosure may be formulated into various forms and administered by a number of different means that will deliver a therapeutically effective amount of the active ingredient. Transdermal administration may involve the use of transdermal patches, hydrogels, topical applications, microneedles, liposomes or iontophoresis or sonophoresis devices. In some aspects transdermal drug transport can be achieved or accompanied by active methods including electrical methods like iontophoresis or electroporation, mechanical administration including microneedles or other methods including jet injectors and ultrasound, photomechanical waves. Passive methods for administration may also be used including but not limited to topical applications like creams, lotions, powders, solutions, pastes, gels, sprays, aerosols, plasters, cataplasms, jellies or oils, suspensions vesicles including liposomes, polymeric nanoparticles, nanoemulsions, prodrug, eutectic systems or mixed with chemical enhancers of uptake. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980), Jeong, W. Y., Kwon, M., Choi, H. E. et al. Recent advances in transdermal drug delivery systems: a review. Biomater Res 25, 24 (2021).

For transdermal administration, penetrants appropriate to the barrier to be permeated are generally included in the preparation. Pharmaceutical compositions adapted for transdermal administration may be formulated as ointments, creams, suspensions, lotions, powders, solutions, pastes, gels, sprays, aerosols, plasters, cataplasms, jellies or oils. In some embodiments, the pharmaceutical composition is applied as a topical ointment or cream.

Generally, a safe and effective amount of a composition of the present disclosure is an amount that would cause the desired therapeutic effect in a subject while minimizing undesired side effects. In various embodiments, an effective amount of a composition of the present disclosure may reduce pain.

The amount of a composition described herein that can be combined with a pharmaceutically acceptable carrier to produce a single dosage form will vary depending upon the subject treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure may be decided by an attending physician within the scope of sound medical judgment.

Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Treating can also include reducing or alleviating discomfort associated with treatment of certain illness. Illustratively, the method of the present invention can be used to relieve nausea, vomiting, pain and/or other discomforts associated with chemotherapy and other treatment regimens used to treat cancer and other illnesses. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician. Relieving may include complete elimination as well as any clinically, quantitatively measurable, or perceptible reduction in the symptoms and/or discomfort.

The compositions of the present disclosure can be used to relieve the symptoms of a variety of diseases, conditions, syndromes, disorders, injuries and other forms of illness. In some aspects the compositions can be used to relieve symptoms like lack of appetite in patients suffering from illnesses, such as cancer and AIDS. Chronic pain and other symptoms associated with neuropathy may also be relieved with the method of the present invention. In some aspects the composition can be used to treat spasticity and other symptoms in patients suffering from conditions including multiple sclerosis or spinal cord injury. In some aspects the compositions of the current disclosure can also be used to relieve symptoms associated with dystonia and malignant tumors. In some aspects the compositions can also be used to relieve symptoms of stroke, head injuries, neurodegenerative disorders, and other conditions, diseases, and disorders associated with the N-methyl-D-aspartate receptor. The compositions of the present disclosure can also be used to treat any condition generally known in the art to be responsive to cannabinoids including but not limited to pain, inflammation, involuntary or continual muscular contractions, muscle twisting and twitching. In some aspects, the pain can be a result of underlying conditions like rheumatoid arthritis and joint pain secondary to other autoimmune diseases, such as inflammatory bowel disease, multiple sclerosis and systemic lupus erythematosus. In some aspects the compositions of the current disclosure can be useful to relieve pain and other conditions associated with deeper tissues, such as peripheral nerves, muscles and synovial tissues. Examples of conditions associated with deeper tissues responsive to cannabinoids include: peripheral neuropathic pain, including but not limited to, the peripheral neuropathic pain associated with diabetic neuropathy, ankylosing spondylitis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, osteoarthritis, rheumatoid osteoarthritis, synovitis and juvenile rheumatoid arthritis. The mechanism by which symptoms are relieved is not particularly critical to the practice of the present invention. Illustratively, symptoms can be relieved by directly treating the underlying illness or by blocking the biological pathways by which the illness produces the symptoms.

In some aspects the compositions of the current disclosure can be used as one or more of an anti-inflammatory, anti-convulsant, anti-psychotic, antioxidant, neuroprotective, anti-cancer, such as melanoma, or immunomodulatory agent.

Administration of a composition of the present disclosure can occur as a single event or over a time course of treatment. For example, a composition of the present disclosure may be administered daily, weekly, bi-weekly, or monthly. For treatment of acute conditions, the time course of treatment may be from at least one day to at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities.

### EXAMPLES

### Example 1

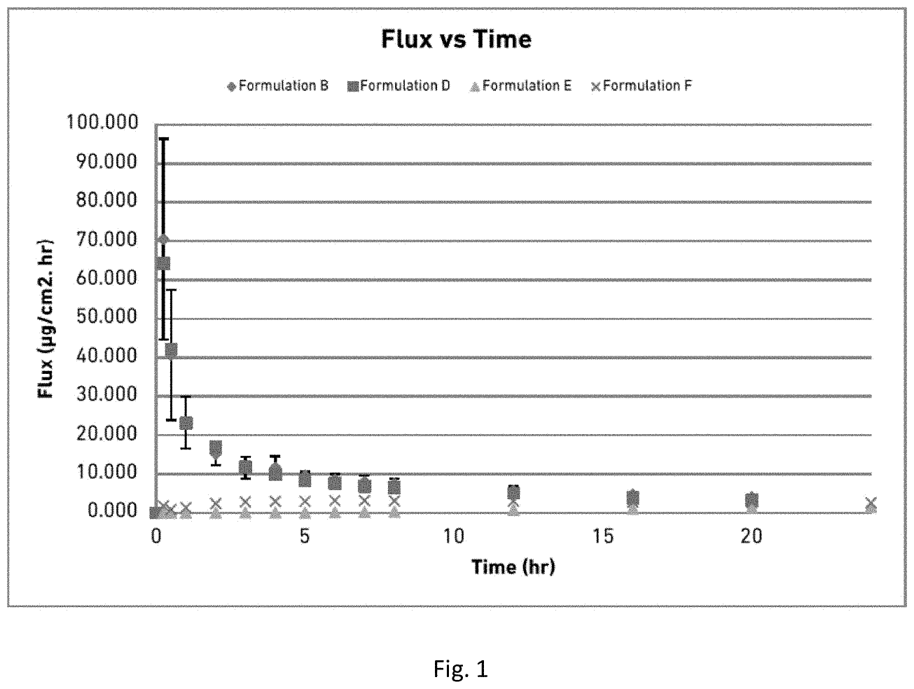

Four formulations, as defined in Table 1, were evaluated for their in vitro permeation performance using a model transdermal drug diffusion system known as a Franz diffusion cell equipped with Strat® M membrane. This membrane is generally regarded as a model for human skin. The receiver compartment contained an aqueous system that was determined to provide infinite sink condition for the cannabidiol. As shown in FIG. 1, formulations B and D have minimal lag time as CBD appears in the receiver media for the first time point at 15 minutes. The flux slowly falls as time progresses and attains steady state condition. The flux for formulations B and D is significantly higher than that observed for formulations E and F.

TABLE 1

| | Formulations | | | |
|---|---|---|---|---|
| Excipient | B | D | E | F |
| Oleyl Alcohol | 0 | 18 | | |
| Isopropyl Myristate | 20 | 20 | 20 | |
| Caprylocaproyl polyoxyl 8 glycerides | 16 | 18 | 21 | |
| diethylene glycol monoethyl ether | 10 | 0 | 10 | |
| propylene glycol monocaprylate | 8 | 6 | 10 | |
| oleyl polyoxyl 6 glycerides | 5 | 0 | 6 | |
| Phosphatidyl choline | 8 | 6 | 0 | |
| Chamomille | 3 | 2 | 3 | |
| polyethylene oxide/polypropylene | 20 | 20 | 20 | |
| oxide/polyethylene oxide glycol | | | | |
| block copolymer | | | | |
| CBD | 10 | 10 | 10 | 10 |
| Medium chain triglycerides | | | | 90 |
| | 100 | 100 | 100 | 100 |

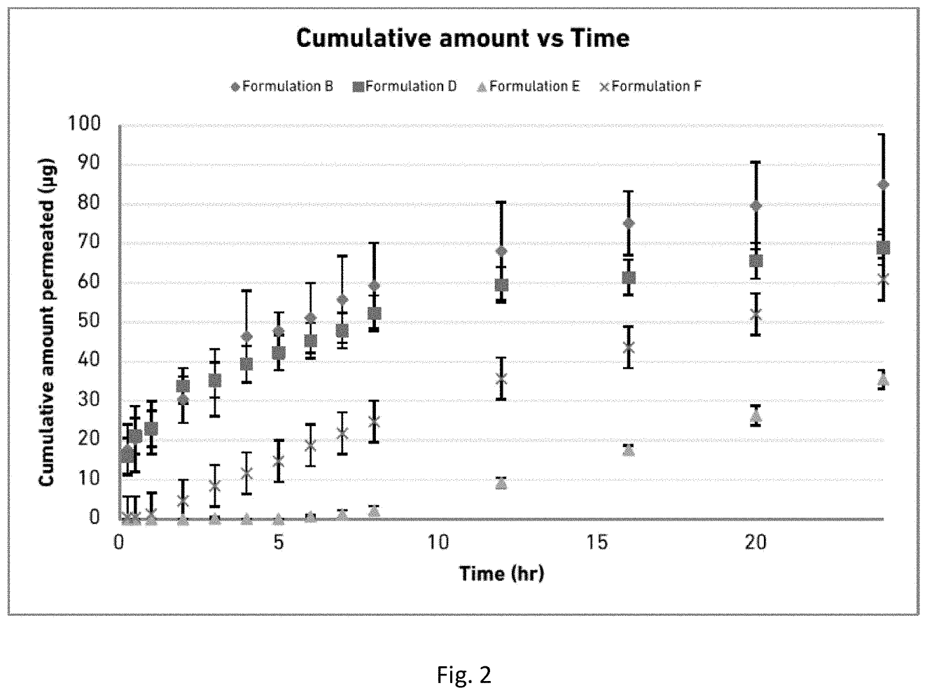

FIG. 2 shows the cumulative amount of CBD that permeated the membrane over time for formulations B, D, E and F. The cumulative amount of CBD permeated through Strat® M membrane in 8 hours for formulations B and D is about 25 times greater than formulation E and about 2 times greater than formulation F. Both formulations B and D successfully enhance the permeation of CBD through Strat® M membrane.

The concentration of CBD in the receiver media at each time point for all the formulations was <0.02340 mg/ml (i.e. 10% of saturation solubility of CBD in receiver media). Hence it appears that sink conditions were maintained during transdermal diffusion study for all the formulations tested.

What is claimed is:

1. A transdermal cannabinoid composition, the composition comprising up to about 20% of one or more cannabinoid, from about 4 to about 15% phosphatidyl choline, and no fatty acids.

2. The transdermal cannabinoid composition of claim 1, wherein the composition comprises up to about 15% cannabinoid.

3. The transdermal cannabinoid composition of claim 2, wherein the composition comprises about 10% cannabinoid.

4. The transdermal cannabinoid composition of claim 1, wherein the cannabinoid is CBD.

5. The transdermal cannabinoid composition of claim 1, wherein the composition comprises about 5 to about 12% phosphatidyl choline.

6. The transdermal cannabinoid composition of claim 1, wherein the composition further comprises isopropyl myristate.

7. The transdermal cannabinoid composition of claim 1, wherein the composition further comprises glycerides.

8. The transdermal cannabinoid composition of claim 7, wherein the glycerides are caprylocaproyl polyoxyl 8 glycerides.

9. The transdermal cannabinoid composition of claim 7, wherein the glycerides are oleyl polyoxyl 6 glycerides.

10. The transdermal cannabinoid composition of claim 1, wherein the composition further comprises PEG or PEG/PPG/PPE copolymers or PPG/PEG/PPG copolymers.

11. The transdermal cannabinoid composition of claim 1, wherein the composition further comprises chamomile or terpenoid blends.

12. The transdermal cannabinoid composition of claim 1, wherein the composition further comprises propylene glycol monocaprylate.

13. The transdermal cannabinoid composition of claim 1, wherein the composition further comprises diethylene glycol monoethyl ether.

14. The transdermal cannabinoid composition of claim 1, wherein the composition further comprises oleyl alcohol.

15. The transdermal cannabinoid composition of claim 1, wherein the composition comprises up to about 20% oleyl alcohol, up to about 30% glycerides, up to about 15% propylene glycol monocaprylate, up to about 5% chamomile, and up to about 30% PEG or PEG/PPG/PEG (polyethylene oxide/polypropylene oxide/polyethylene oxide) block copolymers.

16. A method of transdermally delivering a cannabinoid, the method comprising applying a composition of claim 1 to a subject.
